(19) United States
(12) Patent Application Publication
(10) Pub. No.: US 2013/0345638 A1
(43) Pub. Date: Dec. 26, 2013

(54) MULTIPLE DRUG DELIVERY DEVICE

(71) Applicant: FluGen, Inc., Madison, WI (US)

(72) Inventors: Charles R. Heidenreich, McFarland, WI (US); Reidar S. Aamotsbakken, Madison, WI (US)

(21) Appl. No.: 13/926,082

(22) Filed: Jun. 25, 2013

Related U.S. Application Data
(60) Provisional application No. 61/663,915, filed on Jun. 25, 2012.

(51) Int. Cl. A61M 37/00; A61M 5/315; A61M 5/24

(52) U.S. Cl. CPC A61M 37/0015; A61M 5/2448; A61M 5/31501
USPC 604/173; 604/191; 604/234

(57) ABSTRACT

A drug delivery device is provided. The device is a wearable active transdermal drug delivery device which facilitates drug delivery using one or more vaccine cartridges having microneedles as the point of drug delivery. The device may be used to perform multiple vaccine deliveries to the intradermal layer of a patient.

### CROSS-REFERENCE TO RELATED PATENT APPLICATION

This patent application claims the benefit of U.S. Provisional Patent Application No. 61/663,915, filed Jun. 25, 2012.

### BACKGROUND OF THE INVENTION

The present invention relates generally to the field of drug delivery devices. The present invention relates specifically to wearable active drug delivery devices which facilitate drug delivery using one or more drug cartridges having microneedles as the point of drug delivery.

### BRIEF DESCRIPTION OF THE DRAWINGS

This application will become more fully understood from the following detailed description, taken in conjunction with the accompanying figures, wherein like reference numerals refer to like elements in which:

FIG. 1 is a bottom view of the device prior to actuation;

FIG. 2 includes a cross-sectional view of a drug delivery cartridge prior to actuation;

FIG. 3 includes a cross-sectional view of a drug delivery cartridge following actuation and during drug delivery; and

FIG. 4 includes a cross-sectional view of a drug delivery cartridge after needle retraction.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

A multi-drug delivery device stores individual drugs, pharmaceuticals, hormones, vaccines, or nutrients in separate containers for storage and administration. In a typical embodiment, the drugs are therapeutic or prophylactic vaccines. The vaccines may be, for example, the individual monovalent vaccines of the DENVax vaccine. In another example, the drugs are a combination of monovalent and polyvalent vaccines for the seasonal influenza, pneumonia, chicken pox and shingles delivered from individual fluid containers. The device is employed to deliver the contents to discrete but nearby areas of the intradermal layers of skin.

The delivery of vaccines to the intradermal layers may elicit a more robust immune response compared to a standard needle and syringe delivery to the muscle or subcutaneous layer. For example, simultaneous but independent delivery of the dengue monovalent vaccines, i.e., multi-monovalent delivery, to the dermal layer provides all four monovalent vaccine viruses equal opportunity to replicate thus avoiding the interference observed when delivered in a single tetravalent formulation. Several vaccines including polio and influenza, rabies, yellow fever have been demonstrated to have improved efficacy when delivered intradermally compared to intramuscularly. The multi-monovalent vaccines are injected into the dermal layer of the skin, mimicking the natural route of dengue infection that occurs, for example, from the bite of an infected mosquito.

The vaccine may encompass but is not limited to a live or killed virus, a subunit or conjugate, a viral protein, a DNA plasmid encoding for viral antigens, an anti-sense RNA, a liposome containing viral peptides, a polysaccharide, or any combination of these provided as a liquid formulation. The vaccine may be intended for use in humans or in veterinary applications whether for domestic, dairy or livestock.

For vaccination purposes, skin is a highly accessible organ and represents an effective immune barrier, mainly attributed to the presence of Langerhans cells residing in the epidermis and dendritic cells in the dermis. Skin immunization elicits a broad range of immune responses, including humoral, cellular, and mucosal responses.

No single device exists that allows for the simultaneous delivery of multiple vaccines to the intradermal layers of the skin. Multiple vaccine delivery intramuscularly is currently achieved by sequential delivery, resulting in multiple injections for individual recipients. Such delivery of separate vaccines to neighboring but non-overlapping skin sites could be performed using sequential administrations by needle and syringe. This approach is extremely inefficient and requires additional time for the healthcare worker as well as the patient.

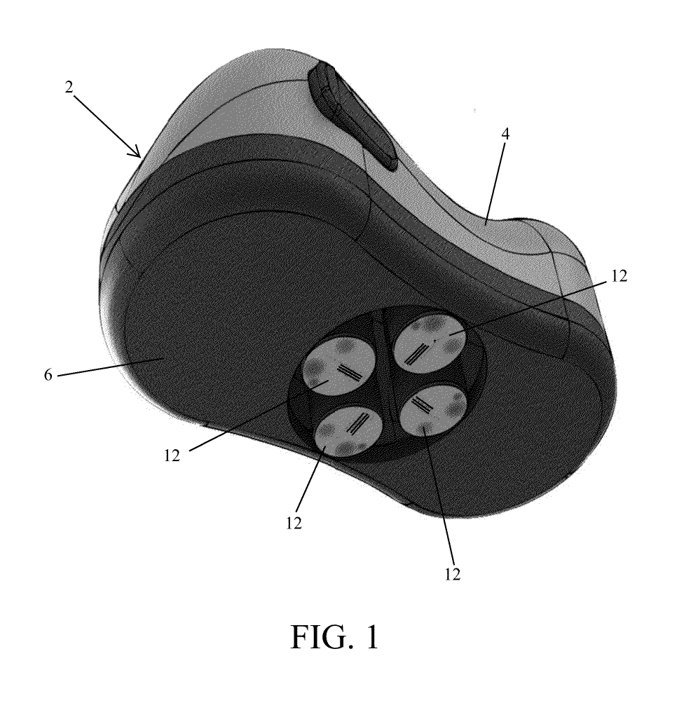

Referring to FIG. 1, a multiple drug delivery device 2 is shown. Delivery device 2 provides concomitant delivery of multiple vaccines from discrete containers through separate needles and to distinct but proximal sites. Delivery device 2 is shown as a housing 4 having a base 6 and four separate drug cartridges 12. Base 6 of delivery device 2 may be provided with an adhesive to secure delivery device 2 to the skin of a patient, thereby tensioning the skin of the patient during needle insertion and drug or vaccine injection. Drug cartridges 12 are inserted and held within housing 4 of delivery device 2. Housing 4 further contains a main spring, trigger, and retraction actuator as discussed in further detail below. In one embodiment, base 6 of delivery device 2 has a footprint of 5 cm by 7 cm.

In other embodiments, the delivery device may be configured with one, two, three, or five or more drug cartridges. In some embodiments, delivery device 2 may be configured to hold four cartridges, but an operator can insert fewer than the maximum number of cartridges and actuate the device. Each drug cartridge 12 stores a drug or vaccine and delivers it, via a dedicated fluid path, to a discrete location in the skin or subcutaneous tissue. Thus, delivery device 2 allows the simultaneous but independent delivery of different vaccines. In some embodiments, each vaccine is a monovalent vaccine so that simultaneous delivery multi-monovalent delivery. In other embodiments, one or more cartridges 12 may contain a combination vaccine while other cartridges 12 contain monovalent vaccines. In a typical embodiment, drug cartridges 12 are spaced at a distance of less than 1 cm from adjacent drug cartridges. In other embodiments, a larger spacing between drug cartridges 12 may be provided.

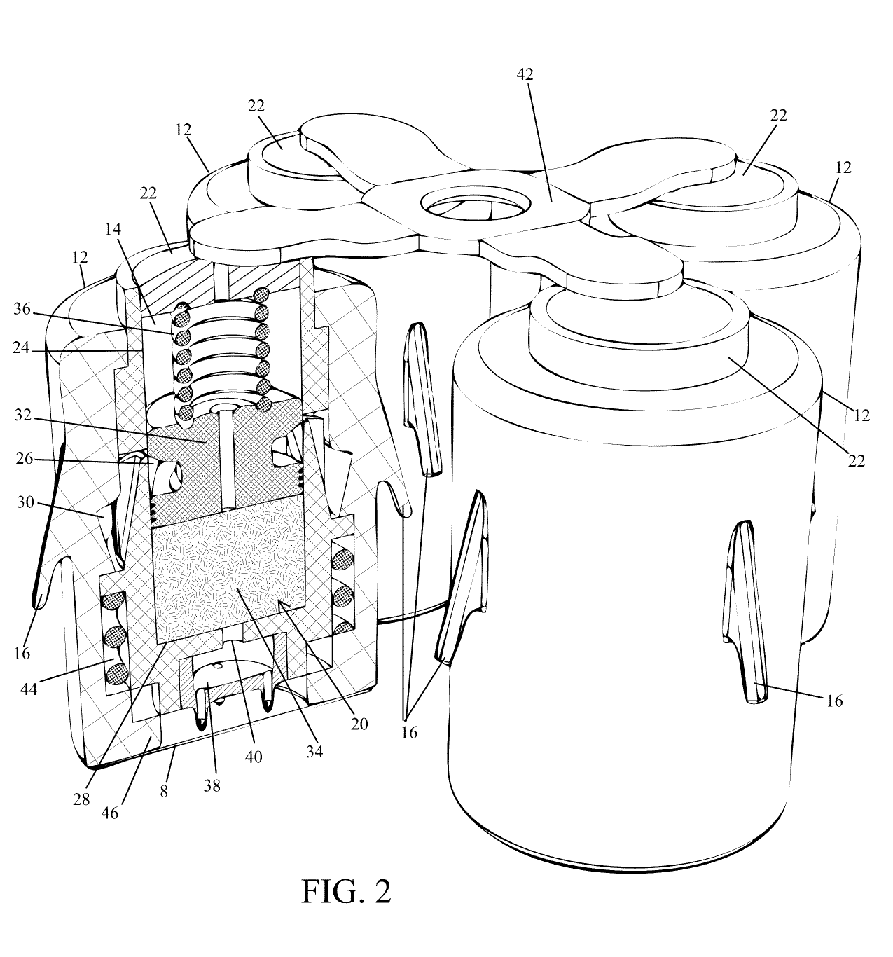

Referring to FIG. 2, a cross-sectional view of a drug cartridge 12 prior to actuation is shown. Drug container 12 is a generally cylindrical cartridge having a cartridge housing 12 defining a central bore 14. Drug cartridges 12 are inserted into the base of the delivery housing prior to patient administration. Drug cartridge 12 may be provided with retaining barbs 16 to lock the drug cartridge 12 into housing 4 and prevent removal after insertion. Drug containers 12 usable with delivery device 2 are preferably cartridges that can be filled and stored outside of delivery device 2 and inserted into the device as needed. In other embodiments, drug cartridges 12 may be secured in delivery device 2 by an adhesive, ultrasonic welding, a retaining ring, etc., and may be installed as part of an integrated manufacturing process and prior to use.

Each cartridge is made up of a primary drug container 20, which includes components that contain and protect the dosage form. Primary drug container 20 moves inside central bore 14 of cartridge housing 12 during needle insertion and retraction. Primary drug container 20 is formed generally as cylinder having top cap 22, cylinder wall 24, plunger interference latches 26, and bottom wall 28. Components of primary drug container 20, for example, top cap 22, bottom wall 28, and microneedle array 38, may be ultrasonically welded to cylinder wall 24. Needle arrays are nested inside the cartridge housing for safety and are protected by a label that is to be removed just prior to actuation. Cartridge housing 12 is provided with one or more recesses 30 sized to receive plunger interference latches 26. A plunger 32 is fitted within cylinder wall 24, defining a fluid chamber 34. A plunger spring 36 is interposed between top cap 22 and plunger 32. Prior to actuation, plunger spring 36 is in a compressed state between top cap 22 and plunger 32, and plunger 32 is held in place by plunger interference latches 26 molded or formed into cylinder wall 24.

Primary drug container 20 is further provided with a microneedle array 38. Microneedle array 38 is an array of one or more hollow microneedles as described in U.S. patent application Ser. No. 13/288,266, which is hereby incorporated by reference in its entirety. Microneedle array 38 may be, for example, an array of polymeric microneedles less than 2 mm in length, and having a hollow lumen and multiple ports located near each tip for fluid delivery. The polymeric material may be a liquid crystal polymer. The microneedles thereby direct vaccine to the immune-rich intradermal layers of the skin leading to improved efficacy. In other embodiments, microneedles may be shorter, e.g. 1 mm in length. In still other embodiments, the microneedles may be longer, e.g. 3 mm, 4 mm, or more, thereby allowing for subcutaneous delivery of the contents of fluid chamber 34 to a patient. Microneedle array 38 is in fluid communication with fluid chamber 34 through opening 40 in bottom wall 28. Prior to actuation, microneedle array 38 is nested inside cartridge housing 12. A protective label 8 seals the microneedle array 38 of primary drug container 20 from contact with external objects.

In another embodiment, cartridge 38 may be provided with one or more metal needles in place of polymeric microneedle array 38. In such an embodiment, the metal needles may be at least 3-4 mm in length, thereby allowing for subcutaneous delivery of the contents of fluid chamber 34 to a patient. In a preferred embodiment using metal needles, each container 12 is provided with a single metal needle. In other embodiments, one or more metal needles may be shorter, e.g. 2 mm, 1 mm, or less, thereby allowing for intradermal delivery of the contents of fluid chamber 34 to a patient.

Geometry of the primary drug container 20, position of plunger latches 26 and dimensions of the plunger 32 may be varied to develop cartridges of different volumes (e.g. 100 μl, 250 μl, and 500 μl). In a preferred embodiment, drug cartridges 12 are designed to be filled through the center of top cap 22 with a septum over-molded into plunger 32 to prevent tampering. The geometry and size of drug cartridges 12 may be selected for compatibility with existing aseptic liquid fill technology. For example, according to ISO 13926-1 the standard dimensions for cartridges and pen systems include outside diameters of 8.65 mm, 10.85 mm, and 10.95 mm.

Primary drug container 20 and the components thereof are formed from suitably inert materials, for example polypropylene, medical grade liquid crystal polymer, stainless steel, glass, etc. The primary drug container, plunger and microneedle array materials a preferably selected based on USP recommendations for primary drug containers in a parenteral device and on materials currently cleared for long-term storage of injectable fluids. In some embodiments, primary drug container 20 may be provided with a glass liner. Vapor barriers may be included if the selected materials exhibit higher than acceptable vapor transmission rates at intended storage conditions. In one embodiment, delivery device 2 may be provided with an aluminum vapor barrier.

Still referring to FIG. 2, one or more main springs 42 are provided. A single main spring 42 may be provided for multiple drug cartridges 12, or independent springs may be provided for one or more drug cartridges 12. As shown in FIG. 2 main spring 42 is a flat cantilever spring element. In a preferred embodiment, main spring 42 is formed from stamped steel. In other embodiments, main spring 42 may be a coil spring, a torsion spring, or another mechanical spring. In still other embodiments, main spring 42 may be a gas spring, or pressure may be applied to top cap 22 by gas discharge or by another pressure source. In a preferred embodiment, main spring 42 is optimized to ensure positive needle penetration into skin. The force required to fully penetrate the skin generally depends on the number and geometry of needles in microneedle arrays 38, and the distance of needle travel from pre-actuated to actuated/penetrated state. In one embodiment, a force of about 26 pounds of force may be used to insert needles of microneedle array 38 into the skin of a patient. In preferred embodiments, main spring 42 is actuated by use of a trigger mechanism, thereby actuating each drug cartridge 12 simultaneously. In other embodiments, a trigger may actuate multiple drug cartridges 12 sequentially rather than simultaneously. In an especially preferred embodiment, a trigger actuation force of less than 4 pound-foot is required to trigger delivery device 2.

In preferred embodiments, microneedle arrays 38 retract fully within device housing 4 after use, thereby preventing injury and contamination from sharps. In the embodiment shown, a retraction spring 44 is provided between cartridge housing 12 and primary drug container 20. As shown, retraction spring 44 is a helical spring oriented coaxially with center bore 14. Prior to actuation, retraction spring 44 is in a generally uncompressed state. Where a retraction spring is provided, main spring 42 additionally must overcome the force of the retraction spring 44. The opening in the base of each cartridge 12, dimension of microneedle arrays 38, and distance between needles and base 6 of housing 4 when delivery device 2 is placed in a retracted state may be designed to minimize or eliminate any human exposure to the needles of microneedle array 38.

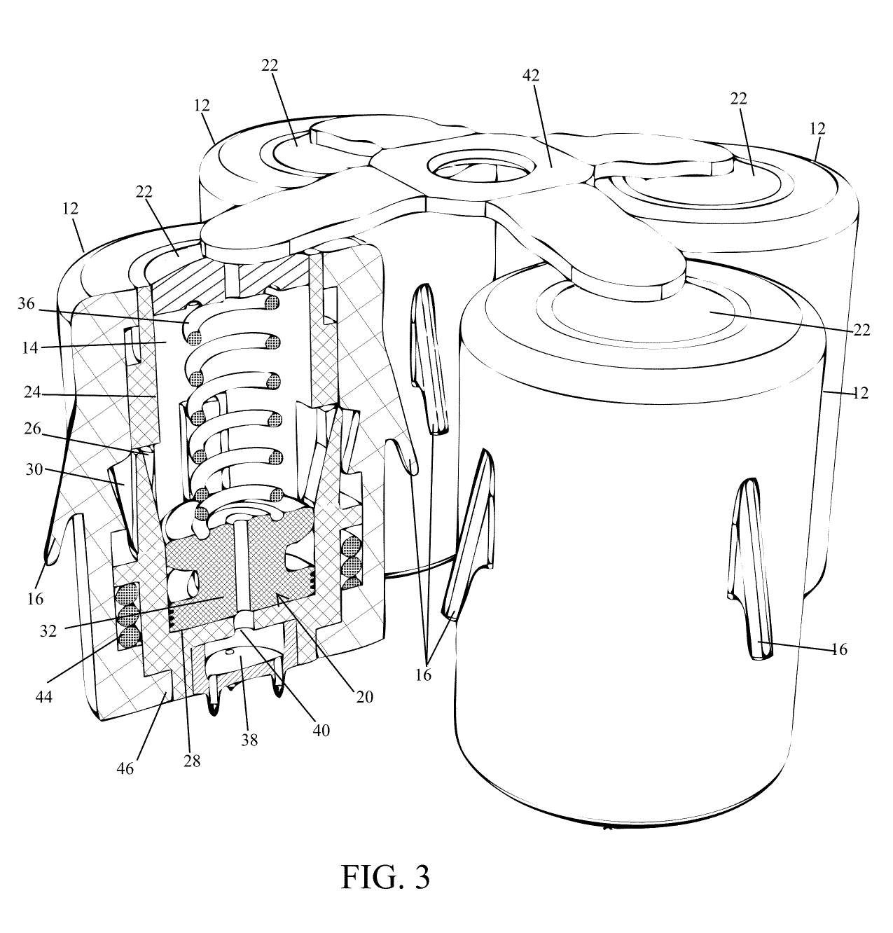

Referring to FIG. 3, upon actuation main spring 42 applies a downward force to top cap 22 of primary drug container 20, thereby driving primary drug container 20 downwards relative to cartridge housing 12 to a mechanical stop 46. The downward motion of primary drug container 20 thereby injects the microneedles of microneedle array 38 into the intradermal layer of a patient. Additionally, downward motion of primary drug container 20 compresses refraction spring 44.

As primary drug container 20 moves downward, plunger interference latches 26 move into alignment with recesses 30 of cartridge housing 12. When the primary drug container 20 is moved inside cartridge housing 12 to the mechanical stop 46, interference latches 26 are forced out of the way into recesses 30 of cartridge housing 12 by the spring force, thereby allowing plunger 32 to travel to the bottom of its stroke within cylinder wall 24. In another embodiment, plunger interference latches 26 may be formed or provided with a spring bias such that the latches 26 rotate into recesses 30, thereby releasing plunger 32 within cylinder wall 24.

Upon release of plunger 32 by plunger interference latches 26, plunger spring 36 moves plunger 32 downwards toward bottom wall 28. As shown, plunger spring 36 is a helical spring oriented coaxially with center bore 14 and retraction spring 44. As plunger 32 moves towards bottom wall 28, plunger 32 forces the contents of fluid chamber 34 through opening 40 and the microneedles of microneedles array 38, and thereby injects the contents of fluid chamber 34 into the skin of a patient. During injection, main spring 42 maintains a downward force on top cap 22 of primary drug container 20. Plunger interference latches 26 move back to their original state on the top side of the plunger once the plunger has completed its stroke.

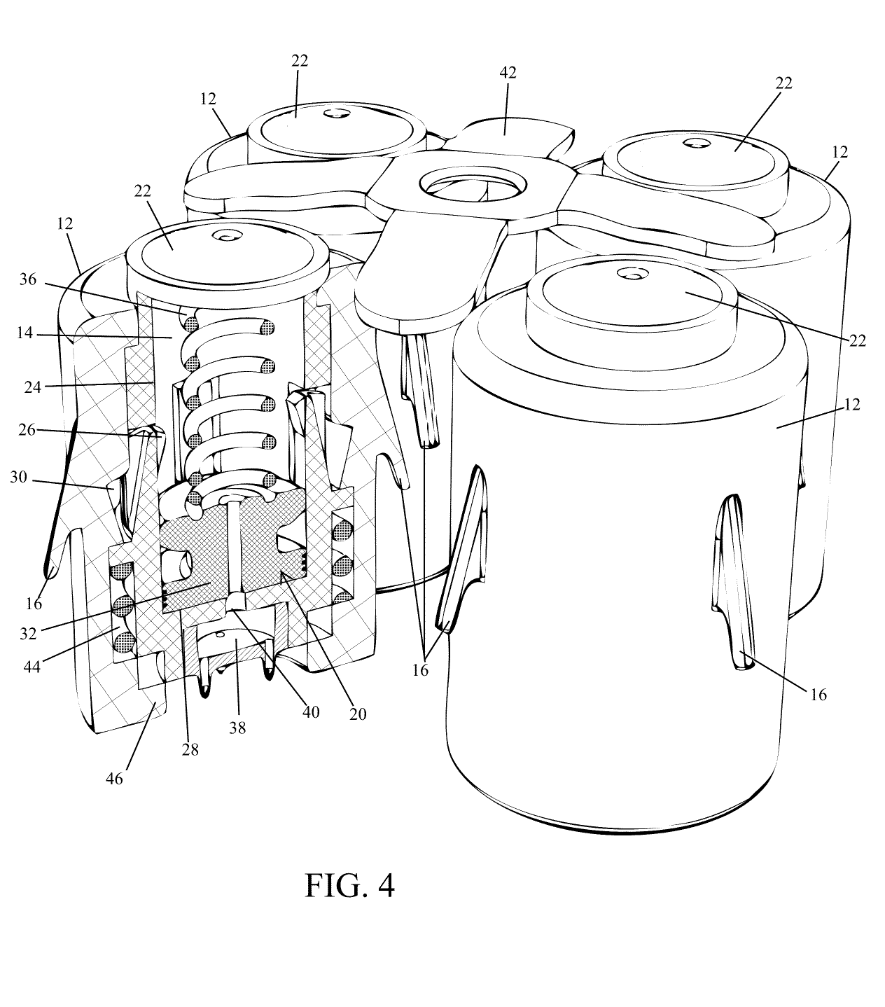

Referring to FIG. 4, upon completion of injection, main spring 42 is removed from contact with top cap 22. In one embodiment, main spring 42 is rotated off of top cap 22 of each drug cartridge 12, thereby releasing primary drug container 20 within center bore 14 of cartridge housing 12. Upon release of the primary drug container 20, retraction spring 44 forces primary drug container 20 upwards, thereby withdrawing the microneedle array 38 from the skin of the patient and retracting the microneedles into cartridge housing 12. The force of retraction spring is generally selected to overcome any plunger interference latches 26 that may remain or protrude into recesses 30 in cartridge housing 12.

Further modifications and alternative embodiments of various aspects of the invention will be apparent to those skilled in the art in view of this description. Accordingly, this description is to be construed as illustrative only. The construction and arrangements, shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. A liquid delivery device, the device comprising:
one or more generally cylindrical walls;
one or more containers, each container comprising a plunger, a plunger spring, a retaining latch, a retraction spring, a liquid storage volume, and one or more needles in fluid communication with the liquid storage volume;
wherein each of the one or more containers is slideably received in each of the one or more generally cylindrical walls;
wherein the retaining latch of each of the one or more containers restricts movement of the plunger of each of the one or more containers when the device is in a first configuration; and
wherein the retaining latch of each of the one or more containers does not restrict movement of the plunger of each of the one or more containers when the device is placed in a second configuration.

2. The device of claim 1, wherein a container further comprises a drug.

3. The device of claim 2, wherein the drug is a vaccine.

4. The device of claim 1, wherein the needles are hollow microneedles.

5. The device of claim 1, wherein the device is moved from the first configuration to the second configuration by application of pressure to a container.

6. A cartridge comprising an outer wall and inner container, the inner container comprising a piston and one or more microneedles, wherein the inner container is slideably received within the outer wall, and wherein the cartridge has a pre-activation configuration, an activated configuration, and a retracted configuration.

7. The cartridge of claim 6, the cartridge further comprising a liquid storage volume in fluid communication with the microneedles, and wherein the piston compresses the liquid storage volume when the cartridge is placed in the activated configuration to thereby channel a liquid through the hollow microneedles.

8. The cartridge of claim 7, wherein the one or more microneedles are configured for intradermal liquid delivery when the cartridge is placed in the activated configuration.

9. The cartridge of claim 7, wherein the one or more microneedles are configured for subcutaneous liquid delivery when the cartridge is placed in the activated configuration.

10. The cartridge of claim 6, wherein the cartridge is moved from pre-activation configuration to the activated configuration by application of pressure to the inner container, and is further moved from the activated configuration the retracted configuration by removal of the pressure from the inner container.

11. The cartridge of claim 6, wherein a plurality of microneedles are provided in an array.

12. The cartridge of claim 6, wherein the one or more microneedles are polymeric microneedles.

13. The cartridge of claim 6, wherein the one or more microneedles are metal needles.

14. The cartridge of claim 6 further comprising a retraction spring, wherein placing the cartridge in the activated configuration compresses the retraction spring.

15. A drug delivery cartridge comprising an outer wall, a retraction spring, and a primary drug container, the primary drug container further comprising liquid storage volume, a piston and piston spring, and one or more microneedles in fluid communication with the liquid storage volume, wherein the primary drug container is slideably received within the outer wall, and wherein the drug delivery cartridge has a pre-activation configuration, an activated configuration, and a retracted configuration.

16. The cartridge of claim 15, wherein the one or more microneedles are configured for fluid communication with the intradermal layers of a patient when the cartridge is placed in the activated configuration.

17. The cartridge of claim 15, wherein the one or more microneedles are configured for fluid communication with the subcutaneous layers of tissue when the cartridge is placed in the activated configuration.

18. The cartridge of claim 15, wherein placing the cartridge in the activated configuration releases one or more retaining latches, thereby allowing the piston to compress the liquid storage volume and thereby channel a liquid through the microneedles.

19. The cartridge of claim 15, wherein the drug delivery cartridge is moved from pre-activation configuration to the activated configuration by an actuation spring, wherein the actuation spring is removably coupled to the primary drug container.

20. The cartridge of claim 19, wherein the cartridge is placed in the retracted configuration by decoupling the actuation spring from the primary drug container.
